(19) United States
(12) Patent Application Publication
(10) Pub. No.: US 2024/0239736 A1
(43) Pub. Date: Jul. 18, 2024

(54) METHOD FOR PRODUCING METHYLENEMALONIC ACID ESTER

(71) Applicant: NIPPON SHOKUBAI CO., LTD., Osaka (JP)

(72) Inventors: Masaru Kirishiki, Suita-shi, Osaka (JP); Hiroki Yamashita, Suita-shi, Osaka (JP); Wataru Ikemoto, Suita-shi, Osaka (JP); Kazuyuki Oshima, Suita-shi, Osaka (JP); Ponnuru Koushik, Florence, KY (US); Hideaki Nagano, Suita-shi, Osaka (JP)

(73) Assignee: NIPPON SHOKUBAI CO., LTD., Osaka (JP)

(21) Appl. No.: 18/024,094

(22) PCT Filed: Sep. 2, 2021
(86) PCT No.: PCT/JP2021/032287

§ 371 Date: Nov. 10, 2023

(30) Foreign Application Priority Data
Sep. 14, 2020 (JP) 2020-153889

(51) Int. Cl. C07C 67/327; B01J 35/61; B01J 37/08

(52) U.S. Cl. CPC C07C 67/327; B01J 35/615; B01J 37/08; B01J 2235/00

(57) ABSTRACT

A method for producing a compound represented by the following Formula (II), the method comprising a step of subjecting a compound represented by the following Formula (I) to heat treatment in the presence of an organic sulfonic acid and an inorganic oxide solid to obtain the compound represented by the following Formula (II), wherein a Hammett acidity function of the inorganic oxide solid is more than −12.0:

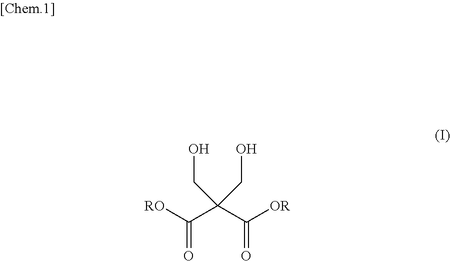

wherein R each independently represents an alkyl group, an alkenyl group, or an aryl group,

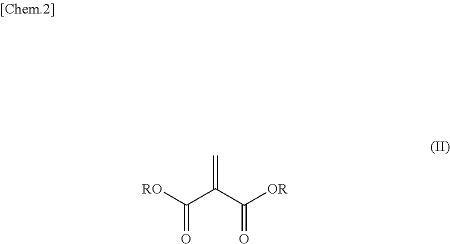

wherein R has the same meaning as R in Formula (I).

### TECHNICAL FIELD

The present invention relates to a method for producing a methylenemalonic acid ester.

### BACKGROUND ART

Methylenemalonic acid esters are expected to be used as a raw material for a coating agent, an adhesive, and the like. For example, as described in Patent Literature 1, a methylenemalonic acid ester (1,1-dicarbonyl-substituted-1-ethylene) is obtained by heat-treating bis(hydroxymethyl)malonic acid ester (1,1-dicarbonyl-substituted-1,1-bis(hydroxymethyl)-methane) having two hydroxymethyl groups at the 1-position of the malonic acid ester. In Patent Literature 1, zeolite is used as a solid acid catalyst during heat treatment.

### CITATION LIST

### Patent Literature

Patent Literature 1: U.S. Pat. No. 9,518,001

### SUMMARY OF INVENTION

### Technical Problem

In the method for obtaining a methylenemalonic acid ester by the heat treatment described above, a bis(hydroxymethyl)malonic acid ester as a reactive substrate needs to be adsorbed on the surface of the solid acid catalyst. When the specific surface area of the solid acid catalyst decreases, the number of pores (adsorption points) to which the reactive substrate can be adsorbed also decreases, resulting in difficulty in maintaining the reaction. That is, when the specific surface area of the solid acid catalyst decreases during the heat treatment, it becomes difficult to continuously perform the heat treatment reaction for a long time.

As a result of studies by the inventors, it has been found that when zeolite is used as the solid acid catalyst as in the method described in Patent Literature 1, the specific surface area is greatly reduced during the reaction.

The present invention has been made in view of the above circumstances, and an object of the present invention is to provide a method for producing a methylenemalonic acid ester capable of sufficiently suppressing a decrease in the specific surface area of an inorganic oxide solid (catalyst) during a reaction of heat-treating a bis(hydroxymethyl)malonic acid ester to obtain a methylenemalonic acid ester.

### Solution to Problem

In view of the above circumstances, as a result of intensive studies, the present inventors have completed the inventions shown in the following [1], [2], and [3]. [1] A method for producing a compound represented by the following Formula (II), the method comprising a step of subjecting a compound represented by the following Formula (I) to heat treatment in the presence of an organic sulfonic acid and an inorganic oxide solid to obtain the compound represented by the following Formula (II), wherein a Hammett acidity function of the inorganic oxide solid is more than −12.0:

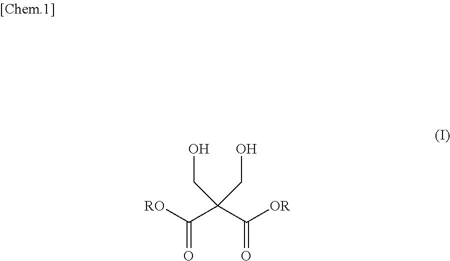

wherein R each independently represents an alkyl group, an alkenyl group, or an aryl group,

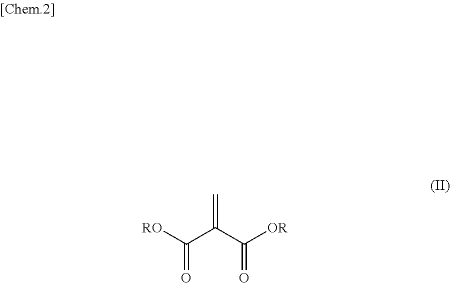

wherein R has the same meaning as R in Formula (I). [2] The production method according to [1], wherein a specific surface area of the inorganic oxide solid before the heat treatment is 2 to 320 m²/g. [3] The production method according to [1] or [2], wherein a ratio of a specific surface area of the inorganic oxide solid after the heat treatment to a specific surface area of the inorganic oxide solid before the heat treatment is 50% or more.

### Advantageous Effects of Invention

According to the present invention, there is provided a method for producing a methylenemalonic acid ester capable of sufficiently suppressing a decrease in the specific surface area of an inorganic oxide solid (catalyst) during a reaction of heat-treating a bis(hydroxymethyl)malonic acid ester to obtain a methylenemalonic acid ester.

### BRIEF DESCRIPTION OF DRAWINGS

FIG. 1 is a schematic view illustrating a reactor X used in Examples.

### DESCRIPTION OF EMBODIMENTS

Hereinafter, an embodiment of the present invention will be described in detail, but the present invention is not limited thereto. Hereinafter, the term “inorganic oxide solid” is also referred to as “catalyst”.

The method for producing a methylenemalonic acid ester of the present embodiment comprises a step of subjecting a compound represented by the following Formula (I) (bis(hydroxymethyl)malonic acid ester) to heat treatment in the presence of an organic sulfonic acid and an inorganic oxide solid to obtain a compound represented by the following Formula (II)(methylenemalonic acid ester).

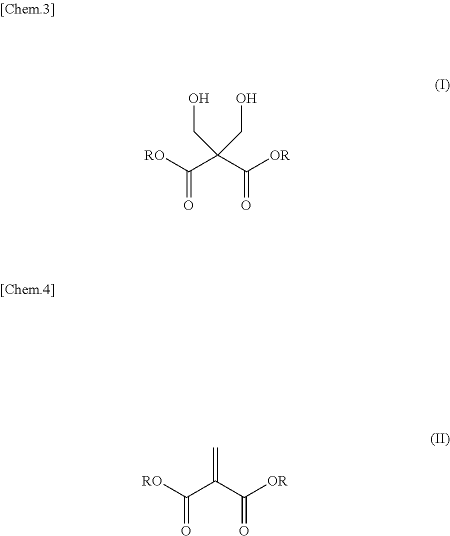

In Formulae (I) and (II), R is each independently represents an alkyl group, an alkenyl group, or an aryl group, and these groups may be linear, branched, or alicyclic.

Examples of the linear alkyl group include a methyl group, an ethyl group, an n-propyl group, an n-butyl group, an n-pentyl group (amyl group), an n-hexyl group, an n-heptyl group, an n-octyl group, an n-nonyl group, and an n-decyl group. The number of carbon atoms of the linear alkyl group can be, for example, 1 to 12.

Examples of the branched alkyl group include an isopropyl group, a sec-butyl group, an isobutyl group, a tert-butyl group, a 1-methylbutyl group, a 1-ethylpropyl group, a 2-methylbutyl group, an isoamyl group, a 1,2-dimethylpropyl group, a 1,1-dimethylpropyl group, a tert-amyl group, a 1,3-dimethylbutyl group, a 3,3-dimethylbutyl group, a 1-methylpentyl group, a 1-methylbutyl group, a 1-ethylbutyl group, a 2-ethylbutyl group, a 2-ethyl-2-methylpropyl group, a sec-heptyl group, a tert-heptyl group, an isoheptyl group, a sec-octyl group, a tert-octyl group, an isooctyl group, a 1-ethylhexyl group, a 1-propylpentyl group, a 2-ethylhexyl group, a 2-propylpentyl group, a sec-nonyl group, a tert-nonyl group, a neononyl group, and a 1-ethylheptyl group. The number of carbon atoms of the branched alkyl group can be, for example, 3 to 12.

Examples of the alicyclic alkyl group include a cyclopentyl group, a cyclohexyl group, a cycloheptyl group, a cyclooctyl group, a methylcyclohexyl group, a cyclohexylmethyl group, an adamantyl group, and a norbornyl group. The number of carbon atoms of the alicyclic alkyl group can be, for example, 5 to 10.

Examples of the linear alkenyl group include a vinyl group, an allyl group, a crotyl group, a 1-butenyl group, a 2-butenyl group, a pentenyl group, a hexenyl group, a heptenyl group, an octenyl group, a nonenyl group, a decenyl group, a dodecenyl group, an octadecenyl group, and an icocenyl group. The number of carbon atoms of the linear alkenyl group can be, for example, 2 to 12.

Examples of the branched alkenyl group include a methallyl group, an isopropenyl group, an isobutenyl group, an isopentenyl group, an isohexenyl group, an isoheptenyl group, an isooctenyl group, an isononenyl group, an isodecenyl group, an isododecenyl group, an isooctadecenyl group, and an isoicocenyl group. The number of carbon atoms of the branched alkenyl group can be, for example, 4 to 12.

Examples of the aryl group include a phenyl group and a naphthyl group. The number of carbon atoms of the aryl group can be, for example, 6 to 12.

R is preferably a linear or branched alkyl group having 1 to 12 carbon atoms, an alicyclic alkyl group having 5 to 10 carbon atoms or an aryl group having 6 to 12 carbon atoms, and more preferably a linear or branched alkyl group having 2 to 10 carbon atoms or an alicyclic alkyl group having 5 to 8 carbon atoms.

Examples of the compound represented by Formula (I) include methylpropyl bis(hydroxymethyl)malonate, di-n-hexyl bis(hydroxymethyl)malonate, dicyclohexyl bis(hydroxymethyl)malonate, diisopropyl bis(hydroxymethyl)malonate, butylmethyl bis(hydroxymethyl)malonate, hexylethyl bis(hydroxymethyl)malonate, di-n-pentyl bis(hydroxymethyl)malonate, ethylpentyl bis(hydroxymethyl)malonate, methylpentyl bis(hydroxymethyl)malonate, butylethyl bis(hydroxymethyl)malonate, di-n-butyl bis(hydroxymethyl)malonate, diethyl bis(hydroxymethyl)malonate (DEM-Diol), dimethyl bis(hydroxymethyl)malonate, di-n-propyl bis(hydroxymethyl)malonate, ethylhexyl bis(hydroxymethyl)malonate, di-n-heptyl bis(hydroxymethyl)malonate, di-n-octyl bis(hydroxymethyl)malonate, di-n-nonyl bis(hydroxymethyl)malonate, and di-n-decyl bis(hydroxymethyl)malonate.

The method for obtaining the compound represented by Formula (I) is not particularly limited, and the compound can be synthesized, for example, by the method described in International Publication No. 2017/197212.

Examples of the compound represented by Formula (II) include a methylenemalonic acid ester obtained using the compound represented by Formula (I) as a raw material. For example, when diethyl bis(hydroxymethyl)malonate (DEM-Diol) is used as a raw material, diethyl methylenemalonate (DEMM) is obtained as the compound represented by Formula (II).

The organic sulfonic acid is not particularly limited as long as it is used as an anionic polymerization inhibitor. Examples of the organic sulfonic acid include alkylsulfonic acids such as methanesulfonic acid, trifluoromethanesulfonic acid and ethanesulfonic acid; and aromatic sulfonic acids such as benzenesulfonic acid and p-toluenesulfonic acid (tosic acid). The addition amount of the organic sulfonic acid may be appropriately adjusted according to the acidity. The addition amount is preferably 0.1 to 5,000 ppm by mass, more preferably 1 to 3,000 ppm by mass, and still more preferably 10 to 2,000 ppm by mass with respect to the total amount of the compound represented by Formula (I), from the viewpoint of balancing storage stability and reactivity.

In the production method of the present embodiment, other anionic polymerization inhibitors may be used in addition to the organic sulfonic acid. As the other anionic polymerization inhibitor, an acid having an acid dissociation constant (pKa) in water of 3 or less is preferable, and an acid having an acid dissociation constant (pKa) of 2.5 or less is more preferable. Specific examples of such an acid include sulfuric acid, sulfurous acid, phosphoric acid, and trifluoroacetic acid.

In the production method of the present embodiment, it is preferable to use a radical polymerization inhibitor or an antioxidant in addition to the organic sulfonic acid. As the radical polymerization inhibitor and the antioxidant, hindered phenols, sulfur-based antioxidants, and phosphorus-based antioxidants are preferable from the viewpoint of suppressing coloring. Specific examples thereof include hindered phenols such as 2,6-di-t-butyl-4-methylphenol (dibutylhydroxytoluene (BHT)), 3-(3,5-di-t-butyl-4-hydroxyphenyl)propionic acid stearate, 2,2′-methylenebis(4-methyl-6-t-butylphenol), tetrakis(methylene-3-(3,5-di-t-butyl-4-hydroxyphenyl)propionate)methane, 4,4′-thiobis(3-methyl-6-t-butylphenol) and 2,5-di-t-butylhydroquinone; sulfur-based antioxidants such as dilauryl thiodipropionate and distearyl thiodipropionate; and phosphorus-based antioxidants such as triphenyl phosphite, tris(nonylphenyl)phosphite, distearyl pentaerythritol diphosphite and tetra(tridecyl)-1,1,3-tris(2-methyl-5-t-butyl-4-hydroxyphenyl)butane diphosphite. The addition amount of the radical polymerization inhibitor or the antioxidant is preferably 50 to 5,000 ppm by mass, more preferably 100 to 3,000 ppm by mass, and still more preferably 200 to 2,100 ppm by mass with respect to the total amount of the compound represented by Formula (I), from the viewpoint of balancing storage stability and reactivity.

The inorganic oxide solid may be a single inorganic oxide or a mixture of a plurality of inorganic oxides. Specific examples of the inorganic oxide solid include Al₂O₃, SiO₂, TiO₂, ZrO₂, CeO₂, ZnO, SiO₂/Al₂O₃, SiO₂/ZrO₂, SiO₂/Y₂O₃, SiO₂/La₂O₃, SiO₂/Ga₂O₃, TiO₂/Al₂O₃, TiO₂/SiO₂, TiO₂/ZrO₂, Al₂O₃/ZrO₂, ZnO/Al₂O₃, TiO₂/B₂O₃, TiO₂/SnO₂, ZnO/SiO₂, ZnO/ZrO₂, Al₂O₃/Bi₂O₃, SiO₂/CaO, and SiO₂/SrO. The inorganic oxide solid is preferably at least one selected from the group consisting of Al₂O₃, SiO₂, TiO₂, ZrO₂, and SiO₂/Al₂O₃ from the viewpoint of more easily maintaining the performance of the catalyst.

The Hammett acidity function (H₀) of the inorganic oxide solid is greater than −12.0. In addition, H₀ may be +1.5 or less. When the Hammett acidity function (H₀) of the inorganic oxide solid is in this range, a decrease in the specific surface area of the catalyst is easily suppressed during a reaction of heat-treating a bis(hydroxymethyl)malonic acid ester to obtain a methylenemalonic acid ester. Thus, the performance as a catalyst is more easily maintained even when such an inorganic oxide solid is continuously used in a reaction of heat-treating a bis(hydroxymethyl)malonic acid ester to obtain a methylenemalonic acid ester.

The specific surface area of the inorganic oxide solid before heat treatment is preferably 2 to 320 m²/g, more preferably 5 to 310 m²/g, and still more preferably 10 to 290 m²/g, from the viewpoint of more easily maintaining a sufficient specific surface area of the inorganic oxide solid after heat treatment of the bis(hydroxymethyl)malonic acid ester.

The ratio of the specific surface area of the inorganic oxide solid after heat treatment to the specific surface area of the inorganic oxide solid before heat treatment is preferably 50% or more, more preferably 60% or more, and still more preferably 70% or more. When the ratio is in this range, the performance as a catalyst is more easily maintained even when the bis(hydroxymethyl)malonic acid ester is continuously used in a reaction of heat-treating the bis(hydroxymethyl)malonic acid ester to obtain a methylenemalonic acid ester.

In the production method of the present embodiment, the heat treatment method is not particularly limited as long as the bis(hydroxymethyl)malonic acid ester, the inorganic oxide solid, and the like are heated to a temperature required for the reaction. For example, a container containing the bis(hydroxymethyl)malonic acid ester, the inorganic oxide solid, and the like may be heated in an oil bath or an electric furnace. The temperature required for the reaction is, for example, 150 to 300° C.

As the method of heat treatment (cracking), for example, the method described in International Publication No. 2017/197212 can also be employed.

The form of the catalyst is not particularly limited, and for example, catalysts having various shapes such as a granular shape or a particle shape formed by crushing, a pellet shape or a honeycomb shape formed by extrusion molding, and a spherical shape formed by rolling granulation can be used. The size of the catalyst is not particularly limited, but for example, a catalyst having a particle size of 0.1 to 5 mm, preferably a particle size of 0.5 to 2 mm can be used as long as the catalyst is in the form of a particle, a sphere, or a pellet. In the case of the honeycomb shape, a catalyst with a honeycomb shape having a total length of 10 to 1,000 mm, preferably 20 to 300 mm, a diameter or an end face width of 5 to 50 mm, preferably 10 to 30 mm, and a porosity of 50 to 80% can be used. In the production method of the present embodiment, the catalyst is preferably crushed and classified by a known method and used as a granular catalyst having a particle size of 0.1 of 2 mm.

The reaction pressure in the production method of the present embodiment can be set from normal pressure to reduced pressure. The lower limit of the reaction pressure is preferably 1 kPa, and more preferably 5 kPa. When the reaction pressure is less than the lower limit, productivity is lowered, which is not desirable. The upper limit of the reaction pressure is preferably 101.3 kPa, and more preferably 30 kPa. By setting the reaction pressure to the upper limit or less, the target product is easily vaporized, and the residence time of the target product on the inorganic oxide solid can be shortened, so that the side reaction can be highly prevented.

In the production method of the present embodiment, heating may be performed in a batch mode or in a continuous mode. When heating is performed in a continuous mode, the feeding rate of the raw material is not particularly limited, but can be, for example, 1 to 10 kg/h per 1 L of the inorganic oxide solid.

### EXAMPLES

Hereinafter, the present invention will be described in more detail with reference to Examples and Comparative Examples. However, this example is one aspect of the present invention, and the present invention is not limited thereto.

### <Reactor X>

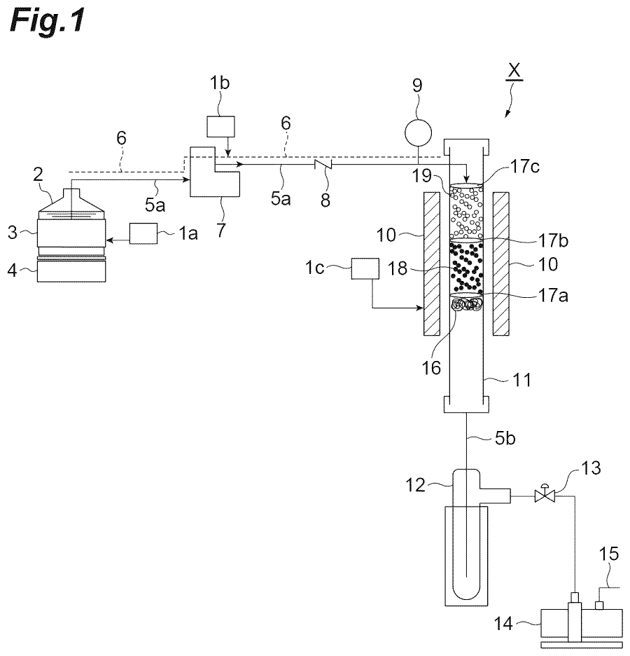

FIG. 1 shows a schematic view of a reactor X used in Examples and Comparative Examples. The reactor X comprises a raw material tank 2, a SUS reaction tube (hereinafter, also simply referred to as “reaction tube”) 11, a reaction solution collection tube 12, a pipe 5a for transferring a raw material from the raw material tank 2 to the reaction tube 11, and a pipe 5b for transferring the reaction solution from the reaction tube 11 to the reaction solution collection tube 12.

The raw material tank 2 comprises a band heater 3 for keeping the raw material tank warm, and a balance 4 for measuring the weight of the raw material in the raw material tank. The band heater 3 is connected to a temperature adjuster 1a.

The pipe 5a comprises a ribbon heater 6 for keeping the pipe warm, a plunger pump 7 for feeding a reaction raw material, a pressure release valve 8, and a pressure gauge 9. The ribbon heater 6 is connected to a temperature adjuster 1b.

The reaction tube 11 is heated by a tubular furnace (electric furnace) 10 connected to a temperature adjuster Ic. Inside the reaction tube 11, a wire mesh demister 16 for holding a catalyst, a quartz wool 17a, a granular catalyst 18, a quartz wool 17b for preventing scattering of the catalyst, glass beads 19 for preheating the raw material, and a quartz wool 17c for preventing scattering of the glass beads are layered in this order.

The reaction solution collection tube 12 is connected to a vacuum pump 14. The vacuum pump 14 comprises a pressure controller 13 and an exhaust line 15.

In Examples and Comparative Examples, evaluation was performed by the reactor X in FIG. 1 using various catalysts (granular catalyst 18).

### <Measurement of Hammett Acidity Function (H₀)>

The acidity functions (H₀) of the granular catalysts used were measured by the following method.

A 50 cm³ Erlenmeyer flask is charged with 10 cm³ of benzene (special grade reagent) that has been dried with a molecular sieve. A granular catalyst is pulverized to 100 mesh or less, then dried at 350° C. for 3 hours and stored in a desiccator, and about 0.1 g of this granular catalyst is placed in the flask. Subsequently, 0.1 mass % benzene solutions are prepared using the indicators described in Table 1, and about 0.1 cm³ of each indicator is added in ascending order of pKa. The maximum acid strength is determined from the pKa of an indicator exhibiting an acidic color first. For example, when there is no acidic color with the 2,4-dinitrotoluene indicator (pKa=−12.0) and there is an acidic color with the dicinnamylideneacetone indicator (pKa=−5.6), the maximum acid strength of the sample is−12.0<H₀<−5.6. The measurement results are shown in Table 2.

TABLE 1

| Indicator | pKa |
|---|---|
| 4-(Phenylazo)diphenylamine | +1.5 |
| Dicinnamylideneacetone | −5.6 |
| 2,4-Dinitrotoluene | −12.0 |

In Examples and Comparative Examples, the specific surface area, the yields of the product (DEMM) and the byproduct (DEM and DEM-Dioxane), and the residual rate of the raw material (DEM-Diol) were measured by the following methods.

### <Specific Surface Area>

The specific surface areas of the granular catalysts before and after heat treatment were determined by nitrogen adsorption at a liquid nitrogen temperature using BELSORP-mini II manufactured by BEL Japan Inc. The specific surface area of the granular catalyst after heat treatment was measured after the following procedures: the catalyst was taken out from the reaction tube after the temperature ofthe catalyst layer, through which the reaction solution from the reaction tube has flowed, was dropped to room temperature; and the collected catalyst was subjected to Soxhlet extraction with tetrahydrofuran (THF) as a solvent. The specific surface areas before and after heat treatment and the ratio of the specific surface area after heat treatment to the specific surface area before heat treatment are shown in Table 3.

The conditions for the Soxhlet extraction are as follows: 110 to 120 mL of THF comprising 100 ppm of methanesulfonic acid (MSA) and 1,000 ppm of dibutylhydroxytoluene (BHT) as a polymerization inhibitor is used for 3 to 3.5 g of the catalyst, and the THF is circulated 15 times at a bath temperature of 95° C.

### <Yields of Product and Byproduct and Residual Rate of Raw Material>

From the reaction solutions obtained in Examples and Comparative Examples, the yield of the product was measured by the following nuclear magnetic resonance spectroscopic (NMR) analysis, the yield of the byproduct was measured by the following gas chromatographic (GC) analysis, and the residual rate of the raw material was measured by the following liquid chromatographic (LC) analysis. When the reaction solution was phase separated, each phase after the phase separation was analyzed.

### <NMR Analysis>

Trimethyl(phenyl)silane as an internal standard substance was added to the oil phase (organic phase), and the mixture was further diluted with chloroform-d. The components of the diluted oil phase were subjected to ¹H-NMR analysis using VNMRS 600 MHz manufactured by Agilent Technologies, Inc. The number of moles of DEMM was calculated based on the obtained chart, and the yield of the product (DEMM) was determined according to the following formula.

$$Yield of DEMM ( mol \% ) = ( number of moles of generated DEMM / number of moles supplied DEM - Diol ) \times 100$$

### <GC Analysis>

Diethylene glycol diethyl ether as an internal standard substance was added to each of the oil phase and the aqueous phase, and the oil phase and the aqueous phase were each further diluted with acetonitrile.

The oil phase components and the aqueous phase components after dilution were analyzed using Nexis GC-2030, manufactured by SHIMADZU Corporation equipped with a FID detector. InertCap 5 manufactured by GL Sciences Inc. was used as a column. The injection temperature was set to 300° C., the detector temperature was set to 320° C., and the thermostatic bath temperature was maintained at 40° C. for 5 minutes. Thereafter, the thermostatic bath temperature was raised to 320° C. at a temperature raising rate of 10° C./min, and analysis was performed. The amount of DEM or DEM-Dioxane contained in all of the oil phase and the aqueous phase was calculated based on the obtained chart. Then, the yields of byproducts (DEM and DEM-Dioxane) were determined according to the following formula.

$$Yield of DEM ( mol \% ) = ( number of moles of generated DEM / number of moles of supplied DEM - Diol ) \times 100$$

$$Yield of DEM - Dioxane ( mol \% ) = ( number of moles of generated DEM - Dioxane \text{} / number of moles of supplied DEM - Diol ) \times 100$$

### <LC Analysis>

Bis(2-ethylhexyl)adipate as an internal standard substance was added to each of the oil phase and the aqueous phase. Further, the oil phase and the aqueous phase were each diluted with acetonitrile as a solvent. The oil phase components and the aqueous phase components after dilution were analyzed using SHIMADZU Prominence equipped with a UV detector. As a column, TSKgel-ODS-100V, manufactured by Tosoh Corporation was used. The analysis was performed at a thermostatic bath temperature of 50° C. The total amount of DEM-Diol contained in all of the oil phase and the aqueous phase was calculated based on the obtained chart. Then, the residual rate of the raw material (DEM-Diol) was determined according to the following formula.

$$Residual rate of DEM - Diol = ( number of moles of DEM - Diol remaining after heat treatment / number of moles of supplied DEM - Diol ) \times 100$$

### Example 1

Activated alumina (model number: KHA-24) manufactured by Sumitomo Chemical Co., Ltd. was crushed, and the crushed product was classified to obtain a classified product having a particle size of 0.70 to 1.00 mm. The obtained classified product was immersed in a tetrahydrofuran solution containing 0.1 wt % of methanesulfonic acid for 5 minutes. After the immersion, the tetrahydrofuran solution containing 0.1 wt % of methanesulfonic acid was removed by decantation, and the classified product was washed three times with acetone. Thereafter, the resulting product was air-dried to obtain a granular catalyst.

The obtained granular catalyst (5 mL) as the granular catalyst 18 was filled on the quartz wool 17a in the reaction tube 11, and the upper part of the granular catalyst 18 was covered with the quartz wool 17b. Further, 4.5 mL of 1 mmp glass beads 19 manufactured by Sogo Laboratory Glass Works Co., Ltd were filled in the reaction tube 11, and the upper part of the glass beads 19 was covered with the quartz wool 17c.

The reaction tube 11 filled with the granular catalyst and the like was placed inside the tubular furnace 10. The vacuum pump 14 was activated, and the reaction pressure was adjusted to 6.66 kPa by the pressure controller 13. Then, the temperature of the tubular furnace (electric furnace) 10 was adjusted to 230° C. by the temperature adjuster 1c. The reaction was started after the pressure of the reaction tube 11 and the temperature of the tubular furnace 10 were stabilized.

DEM-Diol as a raw material was melted by heating, and 1,000 ppm by mass of methanesulfonic acid (MSA) and 2,000 ppm by mass of dibutylhydroxytoluene (BHT) were added to obtain a reaction raw material. The obtained reaction raw material was filled in the raw material tank 2.

In order to prevent the solidification of the reaction raw material present in the raw material tank 2 and the pipe 5a, the temperature of the raw material tank 2 was maintained at about 70° C. by the band heater 3 and the temperature of the pipe 5a was maintained at about 70° C. by the ribbon heater 6. After confirming that the temperature and the pressure of the raw material tank 2 and the pipe 5a were stabilized, the reaction raw material in the raw material tank 2 was fed into the reaction tube 11 at a rate of 0.130 g/min using the plunger pump 7.

The reaction solution was collected in the reaction solution collection tube 12 at a liquid nitrogen temperature for 1 hour from the time point at which 3 hours have elapsed from the start of feeding (start of reaction). The collected reaction solution was returned to room temperature to be liquefied, and separated into an oil phase and an aqueous phase. The results evaluated by the above-described method are shown in Table 3.

### Example 2

Reaction and analysis were performed in the same manner as in Example 1, except that silica-alumina (model number: Neobead SA) manufactured by Mizusawa Industrial Chemicals, Ltd., treated at 1,000° C. for 3 hours, was used as the granular catalyst. The results are shown in Table 3.

### Example 3

Reaction and analysis were performed in the same manner as in Example 1, except that silica (model number: CARiACT Q-50) manufactured by Fuji Silysia Chemical Ltd. was used as the granular catalyst. The results are shown in Table 3.

### Example 4

Reaction and analysis were performed in the same manner as in Example 1, except that silica (model number: CARiACT Q-10) manufactured by Fuji Silysia Chemical Ltd. was used as the granular catalyst. The results are shown in Table 3.

### Example 5

Reaction and analysis were performed in the same manner as in Example 1, except that titania manufactured by Saint-Gobain (model number: ST31119) was used as the granular catalyst. The results are shown in Table 3.

### Example 6

Reaction and analysis were performed in the same manner as in Example 1, except that zirconia manufactured by Saint-Gobain (model number: SZ31163) was used as the granular catalyst. The results are shown in Table 3.

### Comparative Example 1

Reaction and analysis were performed in the same manner as in Example 1, except that ZSM-5 (model number: TC -79MC) manufactured by Tricat Industries, Inc. was used as the granular catalyst.

The results are shown in Table 3.

TABLE 2

| | Catalyst | Acidity function (H₀) |
|---|---|---|
| Example 1 | Al₂O₃ | −5.6 ≥ H₀ > −12.0 |
| Example 2 | SiO₂/Al₂O₃ | −5.6 ≥ H₀ > −12.0 |
| Example 3 | SiO₂ | H₀ > +1.5 |
| Example 4 | SiO₂ | H₀ > +1.5 |
| Example 5 | TiO₂ | +1.5 ≥ H₀ > −5.6 |
| Example 6 | ZrO₂ | H₀ > +1.5 |
| Comparative Example 1 | ZSM-5/Al₂O₃ | −12.0 ≥ H₀ |

TABLE 3

| | Yield (mol %) | | | Residual rate | Specific surface area | |
|---|---|---|---|---|---|---|
| | | DEM- | | of DEM-Diol | (after heat treatment/ | Ratio of specific |
| | DEM | Dioxane | DEMM | (mol %) | before heat treatment) | surface area (%) |
| Example 1 | 4 | 2 | 91 | 3 | 140.9/154 | 91.5 |
| Example 2 | 1 | 3 | 96 | 0 | 8/11 | 72.7 |
| Example 3 | 12 | 7 | 80 | 1 | 67.6/80 | 84.5 |
| Example 4 | 8 | 4 | 87 | 1 | 312/300 | 104 |
| Example 5 | 2 | 10 | 80 | 8 | 33.1/40 | 82.8 |
| Example 6 | 1 | 3 | 89 | 7 | 43.9/55 | 79.8 |
| Comparative | 4 | 3 | 91 | 2 | 66.7/335 | 19.9 |
| Example 1 | | | | | | |

As is apparent from Table 3, in Examples 1 to 6, a decrease in the specific surface area of the catalyst after heat treatment was sufficiently suppressed, whereas in Comparative Example 1, the specific surface area was significantly reduced. Therefore, it is easily estimated that when the reaction is performed using the catalyst of Comparative Example 1 (ZSM-5/Al₂O₃) for a long time, the number of pores (adsorption points) to which the reactive substrate can be adsorbed is insufficient and the reaction cannot be maintained.

### REFERENCE SIGNS LIST

1a, 1b, 1c Temperature adjuster
2 Raw material tank
3 Band heater
4 Balance
5a, 5b Pipe
6 Ribbon heater
7 Plunger pump
8 Pressure release valve
9 Pressure gauge
10 Tubular furnace
11 SUS reaction tube
12 Reaction solution collection tube
13 Pressure controller
14 Vacuum pump
15 Exhaust line
16 Wire mesh demister
17a, 17b, 17c Quartz wool
18 Granular catalyst
19 Glass beads

1. A method for producing a compound represented by the following Formula (II), the method comprising a step of subjecting a compound represented by the following Formula (I) to heat treatment in the presence of an organic sulfonic acid and an inorganic oxide solid to obtain the compound represented by the following Formula (II), wherein
a Hammett acidity function of the inorganic oxide solid is more than −12.0: 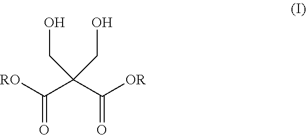
wherein R each independently represents an alkyl group, an alkenyl group, or an aryl group, 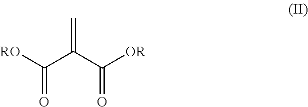
wherein R has the same meaning as R in Formula (I).

2. The production method according to claim 1, wherein a specific surface area of the inorganic oxide solid before the heat treatment is 2 to 320 m²/g.

3. The production method according to claim 1, wherein a ratio of a specific surface area of the inorganic oxide solid after the heat treatment to a specific surface area of the inorganic oxide solid before the heat treatment is 50% or more.

4. The production method according to claim 2, wherein a ratio of a specific surface area of the inorganic oxide solid after the heat treatment to the specific surface area of the inorganic oxide solid before the heat treatment is 50% or more.
